(19) United States
(12) Patent Application Publication
(10) Pub. No.: US 2020/0078160 A1
(43) Pub. Date: Mar. 12, 2020

(54) VASCULAR GRAFTS, METHOD OF MANUFACTURING THEREOF AND ARTICLES COMPRISING THE SAME

(71) Applicants: Sharklet Technologies, Inc., Aurora, CO (US); University of Florida Research Foundation, Inc., Gainesville, FL (US)

(72) Inventors: Chelsea Marie Magin, Denver, CO (US); Anthony B. Brennan, Gainesville, FL (US)

(21) Appl. No.: 16/346,957

(22) PCT Filed: Nov. 7, 2017
(86) PCT No.: PCT/IB2017/056967

§ 371 Date: May 2, 2019

Related U.S. Application Data
(60) Provisional application No. 62/418,402, filed on Nov. 7, 2016.

(51) Int. Cl. A61F 2/06; A61L 27/50; A61L 27/36; A61L 27/24; A61L 27/52; A61L 27/38

(52) U.S. Cl. CPC A61F 2/062; A61L 27/507; A61L 27/3891; A61L 27/24; A61L 27/52; A61L 27/3633

(57) ABSTRACT

Disclosed herein is a method of manufacturing a vascular graft comprising disposing cells of a first cell type on a core having a textured surface, wherein the textured surface comprises a plurality of spaced features, the spaced features being arranged in a plurality of groupings, the groupings of spaced features being arranged with respect to one another so as to define a tortuous path when viewed in a first direction; growing the cells to form a primary cell-seeded construct, wherein the primary cell-seeded construct has a textured inner surface that is a negative image of the textured surface of the core; contacting the primary cell-seeded construct with a second cell type to form a secondary cell-seeded construct; and removing the core to produce the vascular graft.

### CROSS-REFERENCE TO RELATED APPLICATION

This application claims priority to U.S. Provisional Application No. 62/418,402 filed on Nov. 7, 2016, the entire contents of which are hereby incorporated by reference.

### BACKGROUND

This disclosure relates to vascular grafts, methods of manufacture thereof and articles comprising the same.

No clinically available small-diameter vascular grafts have adequate characteristics that are desirable for optimal graft incorporation and healing. The current standard is using autologous vessels such as the saphenous vein and internal thoracic artery as small-diameter grafts. However, this approach is limited by availability and donor site morbidity and failure rates remain at about 50% at 10 years. It is thus not an ideal treatment.

Synthetic grafts have shown success in large-diameter (15 to 35 millimeters (mm)) and medium-diameter arteries (7 to 14 millimeters), but poor patency rates are seen in small-diameter vessels 6 mm) and vascular regeneration with these grafts is not generally viable. Thrombosis and intimal hyperplasia are common causes of graft failure, both of which occur as a result of the absence of endothelial cells lining the lumen of the graft. Infection is another common cause of graft failure, occurring most often in synthetic grafts and allografts. Allografts also have the drawback of variability in tissue availability from cadavers. It is therefore desirable to manufacture a small-diameter vascular graft that is successful in quickly re-establishing vascular flow, promoting endothelialization and does not invoke an immune response.

### SUMMARY

Disclosed herein is a method of manufacturing a vascular graft comprising disposing cells of a first cell type on a core having a textured surface, wherein the textured surface comprises a plurality of spaced features, the spaced features being arranged in a plurality of groupings, the groupings of spaced features being arranged with respect to one another so as to define a tortuous path when viewed in a first direction; growing the cells to form a primary cell-seeded construct, wherein the primary cell-seeded construct has a textured inner surface that is a negative image of the textured surface of the core; contacting the primary cell-seeded construct with a second cell type to form a secondary cell-seeded construct; and removing the core to produce the vascular graft.

Disclosed herein too is a vascular graft comprising a primary cell-seeded construct having a texture on its inner surface, wherein the texture comprises a plurality of spaced features, the spaced features being arranged in a plurality of groupings, the groupings of spaced features being arranged with respect to one another so as to define a tortuous path when viewed in a first direction; and a secondary cell-seeded construct disposed on the primary cell-seeded construct.

Disclosed herein too is a method of using a vascular graft comprising disposing in a body of a living being a vascular graft comprising a primary cell-seeded construct having a texture on its inner surface, wherein the texture comprises a plurality of spaced features, the spaced features being arranged in a plurality of groupings, the groupings of spaced features being arranged with respect to one another so as to define a tortuous path when viewed in a first direction; and a secondary cell-seeded construct disposed on the primary cell-seeded construct, wherein the secondary cell-seeded construct is formed in the body of the living being.

### BRIEF DESCRIPTION OF THE FIGURES

FIG. 1 depicts the method of manufacturing a vascular graft on a textured core;

FIG. 2(A) depicts one type of texture on the surface of the core;

FIG. 2(B) depicts one type of texture on the surface of the core;

FIG. 2(C) depicts one type of texture on the surface of the core;

FIG. 2(D) depicts one type of texture on the surface of the core;

FIG. 3 depicts the average area of the core covered by cells within a given region for static flow; and

FIG. 4 depicts the average area of the core covered by cells within a given region for laminar flow.

### DETAILED DESCRIPTION

A fibroblast is a type of cell that synthesizes an extracellular matrix and collagen to provide the structural framework (stroma) for animal tissue.

Decellularization is the process used in biomedical engineering to isolate an extracellular matrix (ECM) of a tissue from its inhabiting cells, leaving an ECM scaffold of the original tissue, which can be used in artificial organ and tissue regeneration.

Extracellular means “outside the cell” and acellular means “containing no cells”.

Disclosed herein is a method for synthetically manufacturing vascular grafts that have diameters of 5 mm to 35 mm. The method comprises disposing on a textured core (also referred to herein as a substrate), a plurality of cells that are then cultured to form a graft (also referred to herein as a construct). In an embodiment, the graft is an acellular tissue-engineered material made up of an extracellular matrix. The graft may comprise a single layer or multiple layers with one layer disposed atop another layer.

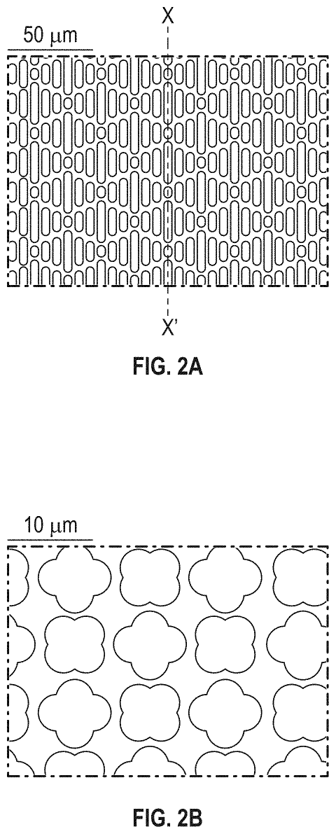

Cell culture is the process by which cells are grown under controlled conditions, generally outside of their natural environment. In practice, the term “cell culture” now refers to the culturing of cells derived from multicellular eukaryotes, especially animal cells. The cultured cells may be grown in a pattern dictated by the texture of the core. In an exemplary embodiment, the first layer of cells to be cultured may be endothelial cells, while the second layer may be smooth muscle cells. Fibroblasts may also be incorporated into this structure. All of these cells are present in the natural blood vessels and will deposit the appropriate extracellular matrix components. The vascular graft aims to rectify the current problem of graft failure due to lack of endothelialization by accelerating the process using the textured micropattern. The textured micropattern is referred to as a SHARKLET® micropattern and is detailed in the FIG. 2(A) and described below.

Disclosed herein is a method for producing a decellularized, tissue-engineered construct in which a core comprising a biocompatible material is contacted with a first suspension comprising a first cell type (e.g., epithelial cells, muscle cells, or the like). The cells of the first type adhere to and cover the core, thereby forming a primary cell-seeded construct. The primary cell-seeded construct is then maintained for a first growth period in an environment suitable for growth of tissue comprising the first cell type to form a primary tissue-engineered construct. In an embodiment, the first cell type comprise epithelial cells.

The core is textured to enable cell growth in a particular direction and this aids in developing strength for the tissue-engineered construct and transfers a pattern to the extracellular matrix proteins deposited by the primary cell type. In another embodiment, the primary cell-seeded construct may be brought in contact with a second suspension comprising a second cell type (e.g., epithelial cells, muscle cells, fibroblasts, macrophages, and the like) which adhere to and adapt to the cell structure of the primary cell-seeded construct, thereby forming a multilayered cell-seeded construct that includes the primary cell-seeded construct (comprising cells of the first cell type) and a secondary cell-seeded construct (comprising cells of the second cell type). This secondary cell-seeded construct is maintained for a second growth period in an environment suitable for growth of the second cell type to form a secondary tissue-engineered construct. The first cell type may be the same or different from the second cell type. The primary cell-seeded construct and/or the secondary tissue-engineered construct may be decellularized as needed.

The formation of the secondary tissue-engineered construct may take place inside or outside of the body of a living being. A seal may be added to the exterior surface of the construct when it is disposed in an artery or vein to inhibit leakage of the bodily fluids that traverse the arteries. Smooth muscle cells such as, for example, fibroblasts and endothelial cells are used in either the primary cell-seeded construct or in the secondary tissue-engineered construct. When the formation of the secondary tissue-engineered construct takes place inside the body of a living being, the core may be made from a biodegradable material. This permits the seeding of the primary cell-seeded construct with the second cell type, while the biodegradable core degrades in the body. The biodegradable core may be partially hollowed out prior to implanting in the body to permit perfusion of biological fluids.

This method is advantageous in that the technique may be used with a wide variety of cell types including stem cells, progenitor cells and patient-specific induced pluripotent stem cells. In one embodiment, this step may take place inside a bioreactor to recreate physiological conditions including temperature, nutrient and flow environments found in vivo.

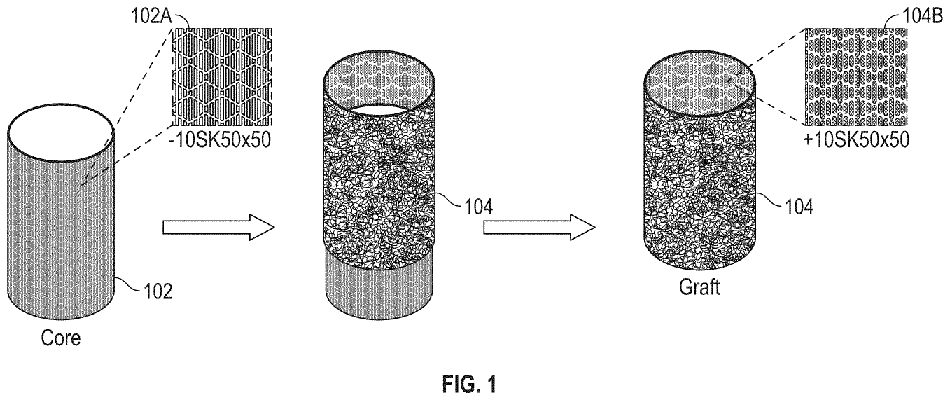

As noted above, the cells to be cultured are disposed on a removable core via a first suspension. The core is sometimes referred to as a mandrel and has its outer surface textured. FIG. 1 depicts the removable core 102 on which is disposed the cells 104 that are to be cultured. In an embodiment, the core is not porous. In another embodiment, the core may be porous. The removable core is generally manufactured from a material that can be removed after the appropriate amount of cell growth has been accomplished on its surface. The cell growth results in the formation of the primary cell-seeded construct (also referred to as a graft). The core 102 has an outer surface that is textured. The texturing serves as a template to direct cell growth in the primary cell-seeded construct. The core 102 can also be separated from primary cell-seeded construct once the desired level of cell growth has occurred. The core 102 can be a disposable core (i.e., it is used once only and then disposed of) or alternatively, it is reusable (i.e., it can be used multiple times). The removal of the core preferably does not damage the primary cell-seeded construct.

In another embodiment (not depicted herein), the core with the primary cell-seeded construct disposed thereon is brought into contact with a second suspension comprising a second cell type (e.g., epithelial cells, muscle cells, fibroblasts, macrophages, or the like) which adhere to and adapt to the cell structure of the primary cell-seeded construct, thereby forming a multilayered cell-seeded construct that includes the primary cell-seeded construct (comprising cells of the first cell type) and a secondary cell-seeded construct (comprising cells of the second cell type). This secondary cell-seeded construct is maintained for a second growth period in an environment suitable for growth of the second cell type to form a secondary tissue-engineered construct. The secondary tissue-engineered construct may then be subjected to decellularization following which it is removed from the core. The secondary tissue-engineered construct may then be used in a vascular graft and implanted in or on a conduit located in the body of a living being.

In an embodiment, when the core 102 is reusable it is in the form of a bladder with an inlet port through which a pressurized fluid is charged to inflate it. Bladders are generally manufactured from an elastomer that can be expanded by a pressurized fluid. The pressurized fluid can be a gas or a liquid. The outer surface of the bladder is textured and the dimensions of the texture can be changed (by varying the amount of bladder inflation) during the culturing of the cells. The use of an inflatable bladder as the core also permits manufacturing primary cell-seeded constructs of different diameters or to change the diameter after the primary cell-seeded construct has begun to form.

After inflating the bladder to a desired level, the desired cells (in the form of a suspension) may be brought into contact with it to facilitate cell growth. Cell growth can take place in a reactor under suitable conditions. When the culturing of the cells is completed and the primary cell-seeded construct is formed, the pressurized fluid is removed from the bladder leaving behind a usable graft that can be stored for future use or alternatively, transferred to the body of a living being.

The bladder may also alternatively be filled with a magnetorheological fluid or an electrorheological fluid that may be subjected to an appropriate magnetic or electrical field to undergo solidification. After forming the primary cell-seeded construct, the applied magnetic or electrical field may be reversed to loosen the core and to remove the primary cell-seeded construct. In an exemplary embodiment, the bladder when inflated has a cylindrical shape or a shape of a conical section.

Suitable elastomers for use in the core are polybutadienes, polyisoprenes, styrene-butadiene rubber, poly(styrene)-block-poly(butadiene), poly(acrylonitrile)-block-poly(styrene)-block-poly(butadiene) (ABS), polychloroprenes, epichlorohydrin rubber, polyacrylic rubber, silicone elastomers (polysiloxanes), fluorosilicone elastomers, fluoroelastomers, perfluoroelastomers, polyether block amides (PEBA), chlorosulfonated polyethylene, ethylene propylene diene rubber (EPR), ethylene-vinyl acetate elastomers, or the like, or a combination thereof. An exemplary elastomer could include a hydrogel with limited swellability to facilitate delivery of nutrients to the cell culture.

In another embodiment, the core 102 can be manufactured from a solid polymer block that is not inflated during the manufacturing of the graft. The solid block polymer may be an organic polymer. Organic polymers used in the core may be selected from a wide variety of thermoplastic polymers, blend of thermoplastic polymers, thermosetting polymers, or blends of thermoplastic polymers with thermosetting polymers. The organic polymer may also be a blend of polymers, copolymers, terpolymers, or combinations comprising at least one of the foregoing organic polymers. The organic polymer can also be an oligomer, a homopolymer, a copolymer, a block copolymer, an alternating block copolymer, a random polymer, a random copolymer, a random block copolymer, a graft copolymer, a star block copolymer, a dendrimer, or the like, or a combination thereof.

Examples of the organic polymers are polyacetals, polyolefins, polyacrylics, polycarbonates, polystyrenes, polyesters, polyamides, polyamideimides, polyarylates, polyarylsulfones, polyethersulfones, polyphenylene sulfides, polyvinyl chlorides, polysulfones, polyimides, polyetherimides, polytetrafluoroethylenes, polyetherketones, polyether etherketones, polyether ketone ketones, polybenzoxazoles, polyphthalides, polyacetals, polyanhydrides, polyvinyl ethers, polyvinyl thioethers, polyvinyl alcohols, polyvinyl ketones, polyvinyl halides, polyvinyl nitriles, polyvinyl esters, polysulfonates, polysulfides, polythioesters, polysulfones, polysulfonamides, polyureas, polyphosphazenes, polysilazanes, styrene acrylonitrile, acrylonitrile-butadiene-styrene (ABS), polyethylene terephthalate, polybutylene terephthalate, polyurethane, polytetrafluoroethylene, fluorinated ethylene propylene, perfluoroalkoxyethylene, polychlorotrifluoroethylene, polyvinylidene fluoride, or the like, or a combination thereof.

Examples of thermosetting polymers suitable for use in the polymeric composition include epoxy polymers, unsaturated polyester polymers, polyimide polymers, bismaleimide polymers, bismaleimide triazine polymers, cyanate ester polymers, vinyl polymers, benzoxazine polymers, benzocyclobutene polymers, acrylics, alkyds, phenol-formaldehyde polymers, novolacs, resoles, melamine-formaldehyde polymers, urea-formaldehyde polymers, hydroxymethylfurans, isocyanates, diallyl phthalate, triallyl cyanurate, triallyl isocyanurate, unsaturated polyesterimides, or the like, or a combination thereof.

Examples of blends of thermoplastic polymers include acrylonitrile-butadiene-styrene/nylon, polycarbonate/acrylonitrile-butadiene-styrene, acrylonitrile butadiene styrene/polyvinyl chloride, polyphenylene ether/polystyrene, polyphenylene ether/nylon, polysulfone/acrylonitrile-butadiene-styrene, polycarbonate/thermoplastic urethane, polycarbonate/polyethylene terephthalate, polycarbonate/polybutylene terephthalate, thermoplastic elastomer alloys, nylon/elastomers, polyester/elastomers, polyethylene terephthalate/polybutylene terephthalate, acetal/elastomer, styrene-maleic anhydride/acrylonitrile-butadiene-styrene, polyether etherketone/polyethersulfone, polyether etherketone/polyetherimide polyethylene/nylon, polyethylene/polyacetal, or the like.

The aforementioned polymers that are used in the core 102 may contain passages in the core through them through which a temperature controlling fluid such as cold water, liquid nitrogen, liquid carbon dioxide, may be transported. The coolants can be used to shrink the diameter of the core 102 after the graft has been manufactured. The reduction in the core diameter can be used to remove the graft from the core 102. In an embodiment, the temperature controlling fluid may both control temperature and supply nutrients for the cells.

In another embodiment, the core 102 may comprise a biodegradable polymer. Polymers that can be used for the pattern or the substrate include biodegradable materials. Suitable examples of biodegradable polymers are as polylactic-glycolic acid (PLGA), poly-caprolactone (PCL), copolymers of polylactic-glycolic acid and poly-caprolactone (PCL-PLGA copolymer), polyhydroxy-butyrate-valerate (PHBV), polyorthoester (POE), polyethylene oxide-butylene terephthalate (PEO-PBTP), poly-D,L-lactic acid-p-dioxanone-polyethylene glycol block copolymer (PLA-DX-PEG), or the like, or combinations comprising at least one of the foregoing biodegradable polymers. The graft 104 after being manufactured can be removed as the core 102 biodegrades. While several of the polymers listed herein (for use in the core) are not biocompatible, they can be rendered biocompatible by increasing the hydrophilicity of the surfaces. This can be done by plasma treating the surface, treating the surface with an electron beam or x-rays, treating the surface with a hydrogel, or the like, or a combination thereof.

The pattern or texture disposed on an outer surface of the core 102 comprises a plurality of spaced features; the features arranged in a plurality of groupings; the groupings of features being arranged with respect to one another so as to define a tortuous path when viewed in a first direction. When viewed in other directions, the groupings of features are arranged to define a linear path. The plurality of spaced features may be projected outwards from a surface or projected into the surface. In one embodiment, the plurality of spaced features may have the same chemical composition as the surface. In another embodiment, the plurality of spaced features may have a different chemical composition from the surface.

The average first feature spacing between the adjacent features is between 1 μm and 100 μm in at least a portion of the surface, wherein said plurality of spaced apart features are represented by a periodic function. It is to be noted that each of the features of the plurality of features are separated from each other and do not contact one another.

In another embodiment, the topography of the texture provides an average roughness factor (R) of from 1 to 50, and preferably 1.5 to 40. As noted above, the pattern is separated from a neighboring pattern by a tortuous path. The tortuous path may be represented by a periodic function. The periodic functions may be different for different tortuous paths. In one embodiment, the patterns can be separated from one another by tortuous paths that can be represented by two or more periodic functions. The periodic functions may comprise a sinusoidal wave. In an exemplary embodiment, the periodic function may comprise at two or more sinusoidal waves.

In another embodiment, when a plurality of different tortuous paths is represented by a plurality of periodic functions respectively, the respective periodic functions may be separated by a fixed phase difference. In yet another embodiment, when a plurality of different tortuous paths is represented by a plurality of periodic functions, the respective periodic functions may be separated by a variable phase difference.

In one embodiment, the plurality of spaced apart features has a substantially planar top surface. In another embodiment, a multi-element plateau layer can be disposed on a portion of the surface, wherein a spacing distance between elements of said plateau layer provide a second feature spacing; the second feature spacing being substantially different when compared to the first feature spacing.

In one embodiment, the pattern comprises a coating layer disposed on said base article. In other words, the coating layer comprises the pattern and is disposed on the base article.

In an embodiment, the features are separated from each other and the average feature spacing is about 1 nanometer to about 500 micrometers. The topography is numerically representable using at least one periodic function; the periodic function being representable by a pathway situated substantially between a plurality of patterns of the spaced apart features.

In one embodiment, the first feature spacing is between 0.5 micrometers (μm) and 5 μm in at least a portion of the surface. In another embodiment, the first feature spacing is between 15 and 60 μm in at least a portion of said surface. As noted above, the periodic function comprises two different sinusoidal waves. In one embodiment, the topography resembles the topography of a shark-skin (e.g., a Sharklet). In another embodiment, the pattern comprises at least one multi-element plateau layer disposed on a portion of the surface, wherein a spacing distance between elements of the plateau layer provides a second feature spacing; the second feature spacing being substantially different when compared to said first feature spacing.

In one embodiment, a sum of a number of features shared by two neighboring groupings is equal to an odd number. In another embodiment, a sum of a number of features shared by two neighboring groupings is equal to an even number.

The number of features in a given pattern can be odd or even. In one embodiment, if the total number of features in a given pattern are equal to an odd number, then the number of shared features are generally equal to an odd number. In another embodiment, if the total number of features in a given pattern are equal to an even number, then the number of features in the given pattern are equal to an even number. The texture or pattern may be embossed on the core.

The texture or pattern can comprise features having different geometries. The FIGS. 2(b), 2(c) and 2(d) show features that comprise circles, sections of circles (e.g., semi-circles, quarter-circles), triangles, and the like.

In one embodiment, a repeat unit can be combined with a neighboring repeat unit so as to produce a combination of spaced apart features that have a geometry that is described by Euclidean mathematics. As can be seen in the FIGS. 2(c) and 2(d), the respective repeat units can be combined to produce different geometries. For example, in the FIG. 2(d), the repeat unit can be combined with a single neighboring repeat unit to produce a diamond shaped geometry. Similarly, 3 or more neighboring repeat units can be combined to produce a rhombohedral, while six repeat units can be combined to produce a hexagon. Thus repeat units may be combined to produce structures whose geometries can be described by Euclidean mathematics.

In one embodiment, the spaced features can have irregular geometries that can be described by non-Euclidean mathematics. Non-Euclidean mathematics is generally used to describe those structures whose mass is directly proportional to a characteristic dimension of the spaced feature raised to a fractional power (e.g., fractional powers such as 1.34, 2.75, 3.53, or the like). Examples of geometries that can be described by non-Euclidean mathematics include fractals and other irregularly shaped spaced features.

In one embodiment, spaced features whose geometries can be described by Euclidean mathematics may be combined to produce features whose geometries can be described by non-Euclidean mathematics. In other words, the groupings of features can have dilational symmetry. The fractal dimension can be measured perpendicular to the surface upon which the features are disposed or may be measured parallel to the surface upon which the features are disposed. The fractal dimensions are measured in the inter-topographical gaps.

In one embodiment, the fractal dimensions can have fractional powers of about 1.00 to about 3.00, specifically about 1.25 to about 2.25, more specifically about 1.35 to about 1.85 in a plane measured parallel to the surface upon which the features are disposed. In another embodiment, the fractal dimensions can have fractional powers of about 1.00 to about 3.00, specifically about 1.25 to about 2.25, more specifically about 1.35 to about 1.85 in a plane measured perpendicular to the surface upon which the features are disposed.

In yet another embodiment, the fractal dimensions can have fractional powers of about 3.00 to about 4.00, specifically about 3.25 to about 3.95, more specifically about 3.35 to about 3.85 in a plane measured perpendicular to the surface upon which the features are disposed. In other words, the tortuous path or the surface of each feature may be textured with features similar to those of the pattern (albeit on a smaller scale), thus creating micro-tortuous paths and nano-tortuous paths within the tortuous path itself.

In yet another embodiment, the spaced features may have multiple fractal dimensions in a direction perpendicular to the surface upon which the features are disposed. The spaced features may be arranged to have 2 or more fractal dimensions, specifically 3 or more dimensions, specifically 4 or more dimensions in a direction parallel to the surface upon which the features are disposed.

As will be noted below, the tortuous path may be defined by a sinusoidal function, a spline function, a polynomial function, or the like. The tortuous path generally exists between a plurality of groupings of spaced features and may occasionally be interrupted by the existence of a feature or by contact between two features. The frequency of the intersection between the tortuous path and the spaced feature may be periodic or aperiodic. In one embodiment, the tortuous path may have a periodicity to it. In another embodiment, the tortuous path may be aperiodic. In one embodiment, two or more separate tortuous paths never intersect one another.

The tortuous path can have a length that extends over the entire length of the surface upon which the pattern is disposed, if the features that act as obstructions in the tortuous path are by-passed. The width of the tortuous path as measured between two adjacent features of two adjacent patterns are about 10 nanometers to about 500 micrometers, specifically about 20 nanometers to about 300 micrometers, specifically about 50 nanometers to about 100 micrometers, and more specifically about 100 nanometers to about 10 micrometers.

The spaced features have linear paths or channels between them. In one embodiment, the spaced features can have a plurality of linear paths or a plurality of channels between them.

The spaced features can be periodic or aperiodic. As noted above, the spaced features can have different dimensions (sizes). The average size of the spaced features can be nanoscale (e.g., they can be less than 100 nanometers) or greater than or equal to about 100 nanometers. In one embodiment, the spaced features can have average dimensions of 1 nanometer to 500 micrometers, specifically about 10 nanometers to about 200 micrometers, and more specifically about 50 nanometers to about 100 micrometers.

In another embodiment, the average periodicity between the spaced features can be about 1 nanometer to about 500 micrometers. In one embodiment, the periodicity between the spaced features can be about 2, 5, 10, 20, 50, 100 or 200 nanometers. In another embodiment, the average periodicity between the spaced features can be about 2, 5, 10, 20, 50, 100 or 200 nanometers. In another embodiment, the periodicity can be about 0.1, 0.2, 0.5, 1, 5, 10, 20, 50, 100, 200, 300, 400 or 450 micrometers. In yet another embodiment, the average periodicity can be about 0.1, 0.2, 0.5, 1, 5, 10, 20, 50, 100, 200, 300, 400 or 450 micrometers.

In one embodiment, the spaced features can have dimensions of 1 nanometer to 500 micrometers, specifically about 10 nanometers to about 200 micrometers, and more specifically about 50 nanometers to about 100 micrometers.

In one embodiment, each feature of a pattern has at least one neighboring feature that has a different geometry (e.g., size or shape). A feature of a pattern is a single element. Each feature of a pattern has at least 2, 3, 4, 5, or 6 neighboring features that have a different geometry from the feature. In one embodiment, there are at least 2 or more different features that form the pattern. In another embodiment, there are at least 3 or more different features that form the pattern. In yet another embodiment, there are at least 4 or more different features that form the pattern. In yet another embodiment, there are at least 5 or more different features that form the pattern.

In another embodiment, each pattern has at least one or more neighboring patterns that have a different size or shape. In other words, a first pattern can have a second neighboring pattern that while comprising the same features as the first pattern can have a different shape from the first pattern. In yet another embodiment, each pattern has at least two or more neighboring patterns that have a different size or shape. In yet another embodiment, each pattern has at least three or more neighboring patterns that have a different size or shape. In yet another embodiment, each pattern has at least four or more neighboring patterns that have a different size or shape.

In an embodiment, the longitudinal axis XX of the pattern is parallel to a longitudinal axis of the core 102. This may be seen in the FIG. 2A. The longitudinal axis XX of a pattern is the line that divides the pattern into two equal halves and that intersects either 0 or 1 element of the pattern.

The patterned core 102 detailed above is used to culture vascular cells to form the primary cell-seeded construct. In an embodiment, a plurality of layers of cells may be cultured on the outer surface of the patterned core 102 to form primary cell-seeded construct. In an exemplary embodiment, the first layer of cells to be cultured may be endothelial cells, the second layer may be smooth muscle cells. Fibroblasts may also be incorporated into this structure. All of these cells are present in the natural blood vessels and will deposit the appropriate extracellular matrix components. This technique may be used with a wide variety of cell types including stem cells, progenitor cells and patient-specific induced pluripotent stem cells.

The step of culturing the cells into a vascular graft is usually conducted inside a bioreactor to recreate physiological conditions including temperature, nutrient and flow environments found in vivo. After the cells have grown to confluence, deposited extracellular matrix components and formed tissue, the vessels will be decellularized using methods described in “Bioengineered human acellular vessels for dialysis access in patients with end-stage renal disease: two phase 2 single-arm trials, Lawson J H, Glickman M H, et. al., 2016” and the extracellular matrix material may be crosslinked in order to preserve the micropatterned structure imparted by the surface of the core. If the core is biodegradable, then it may be removed by exposing the secondary tissue-engineered construct and to a solution that accelerates degradation but does not disrupt the acellular tissue structure.

In an embodiment, the outer surface of the core may have a thin layer of a hydrogel disposed thereon. The hydrogel may have a thickness of a few nanometers to a few micrometers. In an embodiment, the hydrogel may have a thickness of 2 to 500 nanometers. The hydrogel may contain biological markers to enhance cell growth. A biological marker generally refers to a measurable indicator of some biological state or condition. The term is also occasionally used to refer to a substance whose detection indicates the presence of a living organism.

After the hydrogel is disposed on the surface of the core, the core may be subjected to a suspension containing the first cell-type to form the primary cell-seeded construct and then to a second suspension containing the second cell-type to form the secondary cell-seeded construct. The core may then be removed leaving behind the vascular graft. The vascular graft may then be subjected to decellularization and usage.

Examples of hydrogels are polyacrylamide, polyisopropylacrylamide, hydroxyaklylcellulose (hydroxypropylcellulose, hydroxyethylcellulose, hydroxymethylcelluloses, or the like), ora combination thereof.

The primary cell-seeded construct will have a negative image of the texture on its inner surface. The pattern is detailed above and while a few additional details are provided in the succeeding paragraphs below, major details will not be repeated again in the interest of brevity. In other words, a surface of the graft will have a texture where the average periodicity between the spaced features can be about 1 nanometer to about 500 micrometers. In one embodiment, the periodicity between the spaced features can be about 2, 5, 10, 20, 50, 100 or 200 nanometers. In another embodiment, the average periodicity between the spaced features can be about 2, 5, 10, 20, 50, 100 or 200 nanometers. In another embodiment, the periodicity can be about 0.1, 0.2, 0.5, 1, 5, 10, 20, 50, 100, 200, 300, 400 or 450 micrometers. In yet another embodiment, the average periodicity can be about 0.1, 0.2, 0.5, 1, 5, 10, 20, 50, 100, 200, 300, 400 or 450 micrometers.

In one embodiment, the spaced features can have dimensions of 1 nanometer to 500 micrometers, specifically about 10 nanometers to about 200 micrometers, and more specifically about 50 nanometers to about 100 micrometers.

In one embodiment, each feature of a pattern has at least one neighboring feature that has a different geometry (e.g., size or shape). A feature of a pattern is a single element. Each feature of a pattern has at least 2, 3, 4, 5, or 6 neighboring features that have a different geometry from the feature. In one embodiment, there are at least 2 or more different features that form the pattern. In another embodiment, there are at least 3 or more different features that form the pattern. In yet another embodiment, there are at least 4 or more different features that form the pattern. In yet another embodiment, there are at least 5 or more different features that form the pattern.

In another embodiment, each pattern in the primary cell-seeded construct (and eventually in the vascular graft) has at least one or more neighboring patterns that have a different size or shape. In other words, a first pattern can have a second neighboring pattern that while comprising the same features as the first pattern can have a different shape from the first pattern. In yet another embodiment, each pattern has at least two or more neighboring patterns that have a different size or shape. In yet another embodiment, each pattern has at least three or more neighboring patterns that have a different size or shape. In yet another embodiment, each pattern has at least four or more neighboring patterns that have a different size or shape.

In yet another embodiment, the topography of the texture provides an average roughness factor (R) of from 1 to 50. As noted above, the pattern in the primary cell-seeded construct is separated from a neighboring pattern by a tortuous path. The tortuous path may be represented by a periodic function. The periodic functions may be different for different tortuous paths. In one embodiment, the patterns in the graft can be separated from one another by tortuous paths that can be represented by two or more periodic functions. The periodic functions may comprise a sinusoidal wave. In an exemplary embodiment, the periodic function may comprise at two or more sinusoidal waves.

In another embodiment, when a plurality of different tortuous paths are represented by a plurality of periodic functions respectively, the respective periodic functions may be separated by a fixed phase difference. In yet another embodiment, when a plurality of different tortuous paths are represented by a plurality of periodic functions respectively, the respective periodic functions may be separated by a variable phase difference.

In one embodiment, the plurality of spaced apart features in the primary cell-seeded construct have a substantially planar top surface. In another embodiment, a multi-element plateau layer can be disposed on a portion of the surface, wherein a spacing distance between elements of said plateau layer provide a second feature spacing; the second feature spacing being substantially different when compared to the first feature spacing.

A number of different cell types or combinations thereof may be used to manufacture the primary cell-seeded construct, depending upon the intended function of the tissue-engineered construct being produced. Decellularization of the primary cell-seeded construct may take place prior to or after removal of the core to form a basement membrane. Decellularization is conducted using acids, alkaline treatments, ionic detergents, non-ionic detergents, zwitterionic detergents, or a combination thereof.

The ionic detergent, sodium dodecyl sulfate (SDS), is commonly used because of its high efficacy for lysing cells without significant damage to the ECM. Detergents act effectively to lyse the cell membrane and expose the contents to further degradation. After SDS lyses the cell membrane, endonucleases and exonucleases degrade the genetic contents, while other components of the cell is solubilized and washed out of the matrix. SDS is commonly used even though it has a tendency to slightly disrupt the ECM structure. Alkaline and acid treatments can be effective companions with an SDS treatment due to their ability to degrade nucleic acids and solubilize cytoplasmic inclusions.

A useful non-ionic detergent is Triton X-100, which is popular because of its ability to disrupt the interactions between lipids and between lipids and proteins. Triton X-100 does not disrupt protein-protein interactions, which is beneficial to keeping the ECM intact. EDTA is a chelating agent that binds calcium, which is a useful component for proteins to interact with one another. By making calcium unavailable, EDTA prevents the integral proteins between cells from binding to one another. EDTA is often used with trypsin, an enzyme that acts as a protease to cleave the already existing bonds between integral proteins of neighboring cells within a tissue. Together, the EDTA-Trypsin combination are useful for decellularizing tissues.

Enzymes may also be used in decellularization treatments are used to break the bonds and interactions between nucleic acids, interacting cells through neighboring proteins, and other cellular components. Lipases, thermolysin, galactosidase, nucleases, and trypsin have all been used in the removal of cells. After a cell is lysed with a detergent, acid, physical pressure, etc., endonucleases and exonucleases can begin the degradation of the genetic material. Endonucleases cleave deoxyribonucleic acid (DNA) and ribonucleic acid (RNA) in the middle of sequences. Benzoase, an endonuclease, produces multiple small nuclear fragments that can be further degraded and removed from the ECM scaffold. Exonucleases act at the end of DNA sequences to cleave the phosphodiester bonds and further degrade the nucleic acid sequences.

Enzymes such as trypsin act as proteases that cleave the interactions between proteins. Although trypsin can have adverse effects of collagen and elastin fibers of the ECM, using it in a time-sensitive manner controls any potential damage it could cause on the extracellular fibers. Dispase is used to prevent undesired aggregation of cells, which is beneficial in promoting their separating from the ECM scaffold. Dispase is most effective on the surface of a thin tissue, such as a lung in pulmonary tissue regeneration. To successfully remove deep cells of a tissue with dispase, mechanical agitation is often included in the process.

Collagenase is only used when the ECM scaffold product does not require an intact collagen structure. Lipases are commonly used when decellularized skin grafts are needed. Lipase acids function in decellularizing dermal tissues through delipidation and cleaving the interactions between heavily lipidized cells. The enzyme, α-galactosidase is a relevant treatment when removing the Gal epitope antigen from cell surfaces.

As noted above, a number of different cell types or combinations thereof may be used to manufacture the primary cell-seeded construct, depending upon the intended function of the tissue-engineered construct being produced. Thus, for example, smooth muscle cells and endothelial cells may be employed for muscular, tubular tissue-engineered grafts or constructs (e.g., vascular, esophageal, intestinal, rectal, or ureteral constructs); and epithelial, endothelial, fibroblast and nerve cells may be employed in tissue-engineered constructs for the great variety of tissues in which these cells are found. More generally, any cells may be employed which are found in the natural tissue to which the tissue-engineered construct is intended to correspond. In addition, progenitor cells, such as myoblasts or stem cells, may be advantageously employed to produce their corresponding differentiated cell types in a tissue-engineered construct.

Thus, for example, natural arteries are comprised of endothelial, smooth muscle, and fibroblast cells organized into three layers: the intima, the media and the adventitia. The intima is composed primarily of endothelial cells and has three parts: the endothelium, an intermediate layer, and the internal elastic lamina. The media of small arteries consists of 25 to 40 layers of circumferentially disposed smooth muscle fibers between layers of connective tissue, while the media of veins contain relatively fewer (e.g., 5 to 20) layers of smooth muscle. Fibroblasts appear primarily in the adventitia in vivo and are not major components of the normal intimal or medial layers. Therefore, a vascular tissue-engineered construct will preferably include each of these cell types.

In an embodiment, the extracellular matrix comprises a different cell type than a cell type of the primary cell-seeded construct. In an embodiment, the extracellular matrix comprises an identical cell type as a cell type of the primary cell-seeded construct.

In the formation of the primary construct, endothelial cells, for example, may be seeded directly onto a patterned core to create the luminal surface of the construct. Preferably endothelial cells will be allowed to grow for a first period to and deposit extracellular matrix proteins, which will take on the shape of the pattern on the core and form a primary tissue-engineered construct. Smooth muscle cells, for example, may then be seeded onto the primary cell-seeded tissue construct which is allowed to grow for a second period to form a secondary tissue-engineered construct. A low percentage of fibroblasts may also be included in this secondary construct to increase the strength of the resulting construct. After a second growth period, this will produce a secondary tissue-engineered construct having a layer of smooth muscle cells (and, optionally, fibroblasts) surrounding a layer of endothelial cells.

Preferably, the cells are obtained from a live donor and cultured as a primary cell line. In particular, if the tissue-engineered graft/construct is intended to be implanted into a living host, the cells are preferably harvested from the intended host or a histocompatible donor, thereby minimizing or eliminating the possibility of tissue rejection. For example, the desired cells may be obtained from a biopsy of the patient. Thus, in the case of a patient having a coronary by-pass procedure, a biopsy of an artery (e.g. subclavian, axillary, brachial, radial, iliac, ulnar, femoral, anterior or posterior tibial) or peripheral vein (e.g., cephalic, basilic, saphenous, femoral) may be used to obtain arterial smooth muscle, endothelial and fibroblast cells. Alternatively, in the case of a patient requiring, for example, a liver, pancreatic, ureteral, esophageal, intestinal, rectal or other tissue-engineered implant, appropriate cells may be obtained by biopsies of these tissues. It should also be noted that, although not necessarily preferred, biopsies from tissues or organs which do not correspond to the intended implant, but which are phenotypically similar, may be employed. For example, smooth muscle cells derived from an artery may be employed in producing the smooth muscle layers of a venous, esophageal, intestinal, rectal, cardiac or ureteral tissue-engineered construct.

To obtain cells from a donor, standard biopsy techniques known in the art may be employed. Briefly, a desired tissue is surgically removed and the tissue is minced or homogenized, optionally with protease (e.g., trypsin or collagenase) treatment, and a suspension of dissociated cells, or small aggregates of cells, is prepared. Optionally, the cells may then be cultured in vitro in a standard cell growth medium until a suitable number or density of cells are obtained. Although cells may be passaged many times in such cultures, such passaging often causes a loss of differentiated phenotype and, therefore, it is preferred that the number of passages be limited to fewer than 5 or, more preferably, fewer than 3. Most preferably, the cells are not passaged at all.

Alternatively, cells may be employed which are derived from an established cell culture line, either derived in a laboratory or purchased from commercial sources (e.g., ATTC, Rockville, Md.). Typically, such cell lines have lost some degree of differentiation and, therefore, they are generally not preferred. When established cell lines are employed, fetal cell lines or progenitor cell lines may be more desirable because such cells are generally more robust. These cells may also be grown in vitro in a standard cell growth medium until a suitable number or density of cells are obtained.

In another embodiment, cells are employed which have been genetically manipulated by the introduction of exogenous genetic sequences, or the inactivation or modification of endogenous sequences. Thus, for example, genes may be introduced to cause the cells to make proteins which are otherwise absent or defective in the host. Alternatively, production of scarce, but naturally occurring and desirable proteins, such as elastin, may be enhanced by appropriate genetic manipulations of the seeded cells. When implanted into a host, tissue-engineered constructs bearing such cells may serve as a production and delivery system for proteins which are otherwise absent, defective, or insufficient in the host. Thus, for example, genetically engineered endothelial cells that secrete tissue plasminogen activator have been seeded onto various synthetic grafts by Shayani and coworkers (Shayani et al. (1994)), and Chen (Chen et al. (1994)) has demonstrated the feasibility of adenovirus-mediated gene transfer into the endothelial cells of autologous vein grafts as a possible method to improve patency.

Alternatively, repression of gene expression may also be used to modify antigen expression on the surface of seeded cells and tissue constructs, thereby modifying the host's immune response so that cells are not recognized as foreign. Thus, for example, cells incapable of producing one or more MHC proteins, or incapable of loading MHC molecules with antigenic peptides, may be employed to reduce the likelihood of tissue rejection. In such cases, immunosuppression may not be needed when a non-autologous tissue-engineered construct is implanted into a host.

In accordance with the disclosure, mammalian cells are seeded onto and within a porous substrate (e.g., the primary cell-seeded construct) from a suspension so that, preferably, they are evenly distributed throughout the substrate at a relatively high density. Preferably, the cell suspensions comprise approximately 1×10⁴ to 5×10⁷ cells/ml of culture medium, preferably 2×10⁶ to 2×10⁷ cells/ml, and more preferably about 5×10⁶ cells/ml. The optimal concentration of cells in a suspension may, of course, vary according to cell type, the propensity of the cells to form aggregates, the growth rate of the cell type, their binding affinity for the substrate used, and the substrate material used. The suspension may be formed in any physiologically acceptable fluid which does not damage the cells or impair their binding ability (e.g., a standard cell growth medium such as DMEM supplemented with 10% fetal bovine serum).

The cells may be seeded onto and within the core by any standard method. For example, in one embodiment, the primary cell-seeded construct is seeded by submersion into a cell suspension for a fixed period of time, and then the primary cell-seeded construct is removed from the suspension and unbound cells are washed away. Alternatively, the primary cell-seeded construct may be seeded with cells using a syringe or other sterile delivery apparatus. In a currently preferred embodiment, the cell suspension is dripped onto the primary cell-seeded construct and subsequently the primary cell-seeded construct is rotated in, for example, a rotating vessel.

A tubular primary cell-seeded construct, for example, as used in making a muscular, tubular tissue-engineered construct (e.g., a vascular construct), may be rotated about its lumenal axis during or after cell seeding to promote even distribution of the cells onto the surface of the substrate. After allowing a period of time for the cells to bind (optionally incubating the cell-seeded primary cell-seeded construct in growth medium for a period), the cell-seeded primary cell-seeded construct may be immersed in culture medium.

The “seeding time,” or the time between initially contacting the mammalian cells with the substrate and later adding medium, may be varied significantly. Seeding times may vary from 10 minutes to over 1 hour. In the present invention, however, particularly when employing the hydrophilic, synthetic polymeric substrates described and disclosed herein, it has been found that substantially shorter seeding times, from 10 to 30 minutes or, more preferably, about 20 minutes, yield high densities of individually seeded cells with reduced formation of cell aggregates. This seeding time is to be distinguished from the “growth periods” discussed below.

As noted above, the substrates of the present invention may be seeded with suspensions comprising a multiplicity of cell types. Thus, for example, a mixture of two or more cell types (e.g., smooth muscle cells and fibroblasts, or smooth muscle cells and endothelial cells) may be seeded onto a substrate simultaneously, or one or more cell types can be seeded first, followed by seeding with one or more additional types before cell-seeded substrate is placed under suitable conditions for a growth period. In either case, this may be regarded as a single “seeding” although several cell types may be seeded in one or more steps.

Thus, as used herein, the “primary cell-seeded construct” is a substrate which has been subjected to a first seeding with at least one cell type, but possibly more than one cell type, but which has not yet been maintained under suitable conditions for a growth period. During the first growth period, the cells of the primary cell-seeded construct grow and reproduce to yield a “primary tissue-engineered construct” in which the cells may or may not have yet reached confluence. This primary tissue-engineered construct may then be seeded a second time, again with one or more suspensions comprising one or more cell types, to form a “secondary cell-seeded construct.” After maintaining the secondary cell-seeded construct under suitable conditions for a second growth period, during which the cells from the second seeding may grow and reproduce, the resulting construct is referred to herein as a “secondary tissue-engineered construct.” In this manner, several different tissue layers may be disposed on the core prior to its removal.

Thus, for example, a vascular tissue-engineered construct may be produced by seeding smooth muscle cells onto the outer surface of a tubular porous substrate to form a primary cell-seeded construct which is maintained for a first growth period to form a primary tissue-engineered construct, and this construct may then be seeded with endothelial cells (and, optionally, fibroblasts) on the lumenal (and, optionally, outer) surface to form a secondary cell-seeded construct, which is maintained under suitable conditions for a second growth period to form a secondary tissue-engineered construct. Similarly, any number of additional constructs (tertiary, and the like) comprising various cell layers or admixtures, can be engineered according to the present invention (e.g., by inserting a vascular tissue-engineered construct into a larger substrate which is seeded with, for example, hepatocytes to form, ultimately, a vascularized liver tissue-engineered construct). In this manner, several layers of different cellular matter may be disposed on the construct prior to the removal from the core.

In an embodiment, the primary cell-seeded may be formed from a first cell-type that comprises endothelial cells, while the secondary cell-seeded construct may be formed from a second cell-type that comprises at least one of endothelial cells, smooth muscle cells, fibroblasts, and macrophages.

In an embodiment, the “primary cell-seeded construct” with or without decellularization may be transplanted or implanted to replace a portion of a vein or an artery. A seal may be attached to the ends of the construct to prevent fluid leakage. The seal may comprise a biodegradable material that degrades after a period of time without any adverse effects. This construct, now situated within an artery or vein, is seeded with endothelial cells (and, optionally, fibroblasts) on the lumenal (and, optionally, outer) surface to form the secondary cell-seeded construct, which is maintained under suitable conditions for a second growth period to form a secondary tissue-engineered construct at location on the vein or artery. In an embodiment, the secondary cell-seeded construct comprises an extra-cellular matrix that contains smooth muscle cells. The extra-cellular matrix forms the majority of the volume of the construct, based on a total volume of the vascular graft.

In another embodiment, the outermost layer of a multilayered tissue-engineered construct may be suitable for handing and for transportation of the graft to a facility where it is used in the body of a living being. It is therefore desirable for the outermost layer of a multilayered tissue-engineered construct to be robust and versatile. It is capable of withstanding elevated temperatures of 20 to 60° C. and protecting other tissues from impact and damage during transportation and handling. In an embodiment, the outermost layer of a multilayered construct may comprise collagen, polylactic acid, or a combination thereof.

Suitable growth conditions and media for cells in culture are well known in the art. Cell culture media typically comprise essential nutrients, but also optionally include additional elements (e.g., growth factors, salts and minerals) which may be customized for the growth and differentiation of particular cell types. For example, “standard cell growth media” include Dulbecco's Modified Eagles Medium, low glucose (DMEM), with 110 mg/L pyruvate and glutamine, supplemented with 10-20% Fetal Bovine Serum (FBS) or 10-20% calf serum (CS) and 100 U/ml penicillin. Other standard media include Basal Medium Eagle, Minimal Essential Media, McCoy's 5A Medium, and the like, preferably supplemented as above (commercially available from, e.g., JRH Biosciences, Lenexa, Kans.; GIBCO, BRL, Grand Island, N.Y.; Sigma Chemical Co., St. Louis, Mo.).

For use in the methods of the present invention, several variations on standard cell growth media have been developed. In particular, when growing smooth muscle cells, it has been found that the inclusion of the streptomycin should be avoided, as this commonly used antibiotic tends to inhibit the development of the desired phenotype in response to externally applied physical forces, such as the pulsatile force of the invention. In addition, for growing any cells which normally produce a substantial collagenous extracellular matrix, an “enhanced cell growth medium” has been developed which comprises standard cell growth medium, as described above, supplemented with 1-10 mM, preferably 5 mM, HEPES buffer; 0.01-0.1 g/L, preferably 0.02-0.06 g/L, Vitamin C; 0.01-0.1 g/L, preferably 0.02-0.06 g/L, proline; 0.01-0.1 g/L, preferably 0.02-0.06 g/L, glycine; 0.01-0.1 g/L, preferably 0.02-0.06 g/L, alanine; and 0.5-5.0 μg/L, preferably 1.0-3.0 μg/L, of a copper salt (e.g., CuSO₄). Because Vitamin C has a half-life of only 6 to 8 hours at 37° C. in culture medium, Vitamin C is preferably replenished daily to enhance collagen synthesis by the cells. In addition, proline, glycine, and alanine are provided in excess to provide adequate amounts of these amino acids for the synthesis of collagen and other extracellular matrix proteins such as elastin. Copper ions are a necessary co-factor for elastin synthesis and, therefore, a source of copper ions (e.g., CuSO₄) is preferably included in media used to grow elastin-rich tissues. For the growth of endothelial cells, it is preferred that CS be used rather than FBS. In addition, growth factors, such as acidic fibroblast growth factor (aFGF), basic fibroblast growth factor (bFGF), platelet-derived growth factor (PDGF), transforming growth factor β (TGF-β), or vascular endothelial cell derived growth factor (VEGF), may be employed at suitable concentrations (i.e., 1-10 ng/ml) to enhance cell growth or differentiation or the secretion of extracellular matrix proteins.

Cells are cultured under sterile conditions in an atmosphere of 5 to 15% or, preferably, 10% CO2 and 90 to 100% or, preferably, 100% humidity in culture medium at or near the body temperature of the species of origin of the cells or the intended host (i.e., body temperature±5° C., preferably ±2° C.). Thus, for example, human cells may be cultured at 32 to 43° C., more preferably 35 to 39° C., and most preferably 37° C. Cell viability may be determined by standard methods (e.g., trypan blue exclusion) known in the art, or by measuring cell attachment to, and the extent of proliferation on, the substrate. Quantitative assessment of in vitro cell attachment and viability may also be assessed using scanning electron microscopy, histology, and the incorporation of radioisotopes (e.g., 3H thymidine) according to art known methods.

To further enhance the attachment of cells to the substrate and/or to each other, various proteins or growth factors may be provided. For example, collagen, elastin, fibronectin, or laminin, may be provided to the substrate or to the growing constructs to promote cell adhesion. Thus, overlaying collagen on a material such as a polyanhydride substrate can increase adhesion of cells such as hepatocytes. Similarly, the substrate or construct can be impregnated with growth factors such as AFGF, bFGF, PDGF, TGF-β, VEGF, and various other angiogenic and/or other bioactive compounds that may be incorporated directly into the substrate or otherwise contacted with the growing cells (e.g., by addition to the cell culture medium). Multiple growth factors have been studied for their mitogenic effects on endothelial and smooth muscle cells (D'Amore and Smith (1993)). For example, aFGF, bFGF, PDGF have been found to stimulate smooth muscle cell proliferation, while bFGF and VEGF stimulate aortic endothelial cell growth. Basic FGF and VEGF have also been shown to bind to the subendothelial extracellular matrix and basement membrane, and are potent angiogenic factors (Edelman et al. (1991); Rogelj et al. (1989)).

The vascular graft will be provided packaged and sterile to a surgeon. The graft can be trimmed by the surgeon to the appropriate length. Multiple diameter grafts can be manufactured by this technique. The graft can then be inserted by the surgeon and held in place by sutures.

In an embodiment, the vascular graft may be loaded with biologically active agents that may provide the graft with disease fighting properties, anti-inflammatory properties, and the like. The vascular graft acts as a carrier, and the biologically active agent is gradually released from the vascular graft after it is implanted into a blood vessel such as an artery or vein. In an embodiment, the vascular graft may be slit along its length and disposed on a blood vessel to reduce volumetric expansion due to aneurysms.

When the vascular graft is infiltrated (i.e., not covalently bonded) with the biologically active agent, the release of the biologically active agent from the drug coating is diffusion controlled. It is generally desirable for the vascular graft to comprise an amount of 5 to 90 wt %, preferably 20 to 75 wt %, and more preferably 30 to 65 wt %, of the biologically active agent based on the total weight of the vascular graft.

The biologically active agent may be added as a surface coating to the vascular graft or alternatively may be dispersed in the extracellular matrix of the vascular graft. When a surface coating is used, the release of the biologically active agent is interfacially controlled. The drug coating may be disposed only on the surface of the features or alternatively on the surface of the tortuous pathway.

Various types of biologically active agents may be used in the vascular graft. The vascular graft may be used to deliver therapeutic and pharmaceutically biologically active agents including anti-analgesic agents, anti-arrhythmic agents, anti-bacterial agents, anti-cholinergic agents, anti-coagulant agents, anti-convulsant agents, anti-depressant agents, anti-diabetic agents, anti-diuretic agents, anti-fungal agents, anti-hypertensive agents, anti-inflammatory agents, anti-malarial agents, anti-neoplastic agents, anti-nootropic agents, anti-Parkinson agents, anti-retroviral agents, anti-tuberculosis agents, anti-tussive agents, anti-ulcerative agents, anti-viral agents, or the like, or a combination comprising at least one of the foregoing therapeutic and pharmaceutically biologically active agents.

Examples of other suitable therapeutic and pharmaceutically biologically active agents are anti-proliferative/antimitotic agents including natural products such as vinca alkaloids (e.g., vinblastine, vincristine, and vinorelbine), paclitaxel, epidipodophyllotoxins (e.g., etoposide, teniposide), antibiotics (e.g., dactinomycin, actinomycin D, daunorubicin, doxorubicin, penicillin V, penicillin G, ampicillin, amoxicillin, cephalosporin, tetracycline, doxycycline, minocycline, demeclocycline, erythromycin, aminoglycoside antibiotics, polypeptide antibiotics, nystatin, griseofulvin, and idarubicin), anthracyclines, mitoxantrone, bleomycins, plicamycin, mithramycin and mitomycin, enzymes (L-asparaginase, which systemically metabolizes L-asparagine and deprives cells which do not have the capacity to synthesize their own asparagine), antiplatelet agents such as G(GP) Ilb/Illa inhibitors and vitronectin receptor antagonists, anti-proliferative/antimitotic alkylating agents such as nitrogen mustards (e.g., mechlorethamine, cyclophosphamide and analogs, melphalan, chlorambucil), ethylenimines and methylmelamines (e.g., hexamethylmelamine and thiotepa), alkyl sulfonates-busulfan, nitrosoureas (e.g., carmustine (BCNU) and analogs, streptozocin), trazenes-dacarbazinine (DTIC), anti-proliferative/antimitotic antimetabolites such as folic acid analogs (e.g., methotrexate), pyrimidine analogs (e.g., fluorouracil, floxuridine, cytarabine), purine analogs and related inhibitors (e.g., mercaptopurine, thioguanine, pentostatin and 2-chlorodeoxyadenosine {cladribine}), platinum coordination complexes (e.g., cisplatin, carboplatin), procarbazine, hydroxyurea, mitotane, aminoglutethimide, hormones (e.g., estrogen), anti-coagulants (e.g., heparin, synthetic heparin salts and other inhibitors of thrombin), fibrinolytic agents (e.g., tissue plasminogen activator, streptokinase and urokinase), aspirin, dipyridamole, ticlopidine, clopidogrel, abciximab, antimigratory, antisecretory (e.g., breveldin), anti-inflammatory: such as adrenocortical steroids (e.g., cortisol, cortisone, fludrocortisone, prednisone, prednisolone, 6α-methylprednisolone, triamcinolone, betamethasone, and dexamethasone), non-steroidal agents (e.g., salicylic acid derivatives such as aspirin, para-aminophenol derivatives such as acetominophen, indole and indene acetic acids (e.g., indomethacin, sulindac, etodalac), heteroaryl acetic acids (e.g., tolmetin, diclofenac, ketorolac), arylpropionic acids (e.g., ibuprofen and derivatives), anthranilic acids (e.g., mefenamic acid, meclofenamic acid), enolic acids (e.g., piroxicam, tenoxicam, phenylbutazone, oxyphenthatrazone), nabumetone, gold compounds (e.g., auranofin, aurothioglucose, gold sodium thiomalate), immunosuppressives (e.g., cyclosporine, tacrolimus (FK-506), sirolimus (e.g., rapamycin, azathioprine, mycophenolate mofetil), angiogenic agents such as vascular endothelial growth factor (VEGF), fibroblast growth factor (FGF), angiotensin receptor blockers, nitric oxide donors, anti-sense oligionucleotides and combinations thereof, cell cycle inhibitors, mTOR inhibitors, and growth factor receptor signal transduction kinase inhibitors, retenoids, cyclin/CDK inhibitors, HMG co-enzyme reductase inhibitors (statins) or protease inhibitors. The biologically active agents may also include cancer inhibitors.

The method detailed herein is advantageous in that the grafts can be customized to be any diameter, depending on the scaffold size used to grow it. The Sharklet-patterned mandrel may be made of a biodegradable material tuned to degrade with timing that imparts texture to the luminal surface of the vascular graft without the need for removal from a patterned rod.

The method and the vascular graft detailed herein is exemplified by the following example.

### Example

Smooth (SM) and micropatterned (+1.7SK2x2, +1.2SK10x5 and +11.4SK50x50) samples were fabricated by casting polydimethylsiloxane elastomer (Xiameter RTV-4234-T2, Dow Corning; PDMSe) against negative silicon wafer molds and adhering these surfaces to silane-treated glass slides.

The nomenclature adopted here (e.g., +1.7SK2x2) should deciphered as follows: The +1.7 indicates the height of the texture above the base surface in micrometers while the SK refers to a Sharklet pattern depicted and described in U.S. Pat. No. 7,143,709 B2 to Brennan et al., and patent application Ser. No. 12/550,870 to Brennan et al. A negative sign (−) preceding the 1.7 would indicate that the texture is below the base surface. The first 2 in SK2x2 stands for the width of each feature in the pattern while the second 2 stands for the spacing between the features in the pattern in micrometers.

The PDMSe samples were cast such that the long axis of the features was aligned with the long axis of the glass slide. All samples were treated with fibronectin (15 μg/mL) and collagen (200 μg/mL) to facilitate cell attachment and mimic ECM composition.

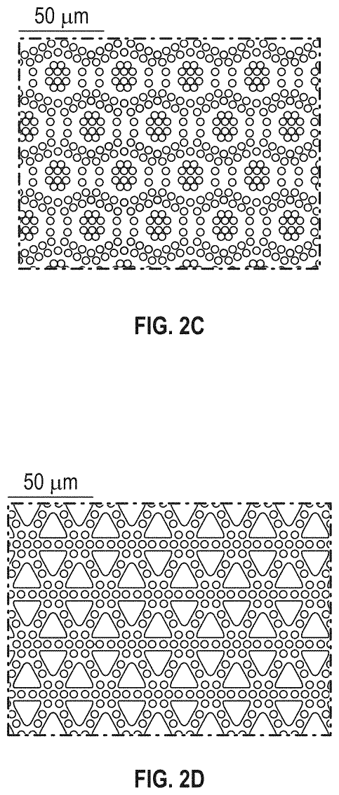

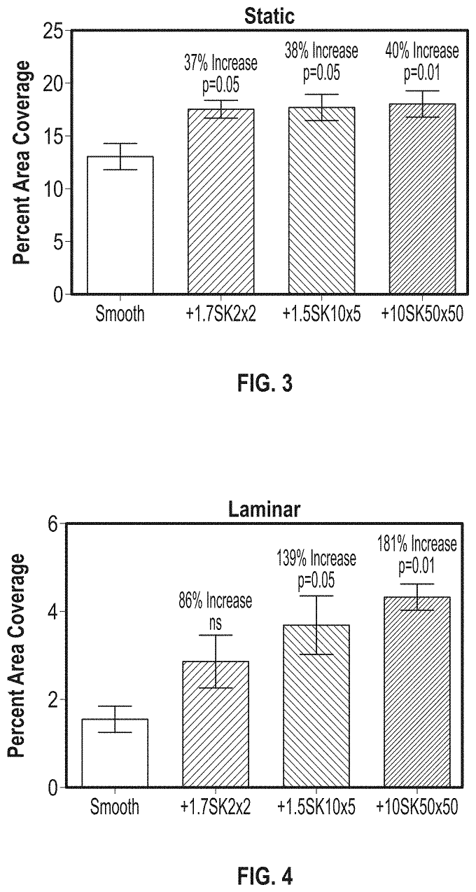

A modified scratch-wound assay was used to investigate cell migration. Briefly, SM PDMSe rectangles (3 mm×25 mm) were placed along the center of the sample to create an artificially wounded area. Human coronary artery endothelial cells (HCAECs; ATCC) were seeded over the entire configuration at 3×10⁴ cells/cm² and maintained in complete vascular endothelial growth media (Vascular Cell Basal Medium, 2% FBS, 5 ng/mL rh VEGF, 5 ng/mL rh EGF, 5 ng/mL rh FGF, 15 ng/mL rh IGF-1, 10 mM L-glutamine, 0.75 Units/mL heparin sulfate, 1 μg/mL hydrocortisone, 50 μg/mL ascorbic acid, and 50 U/ml penicillin/streptomyocin). At ˜80% confluence, PDMSe rectangles were removed to allow cell migration across the empty patterned area. The sample was then either maintained in complete vascular endothelial growth media for 3 days for static experiments (FIG. 3) or for placed in a parallel plate flow chamber (Glycotech, 31-010) where it was subjected to 5 dyne/cm² of shear for 24 hours for laminar flow experiments (FIG. 4). The samples were then stained with CellTracker Orange according to the manufacturer's instructions and fixed with 4% paraformaldehyde at room temperature. Fluorescent microscopy images were taken of the wounded area and the average area covered by cells within this region was calculated using ImageJ software.

Results from static migration showed all three patterns significantly improved cell migration compared to smooth (Dunnett's Test, FIG. 3). The +10SK50x50 micropattern resulted in the highest level of increased migration (40%, p=0.01). Assays conducted under laminar flow revealed differences in percent area coverage for the various Sharklet micropatterns (FIG. 4). Both +1.5SK10x5 and +10SK50x50 increased cell migration significantly compared to smooth, 139%, p=0.05 and 181%, p=0.01, respectively.

In addition, it may be seen that textures with larger spacings between the individual features of the pattern and larger individual feature widths produce larger surface coverage under laminar flow conditions than when under static growth conditions. Thus, for example, features having a width of 3 micrometers or greater, preferably 5 micrometers or greater, and more preferably 10 micrometers or greater with an average feature spacing of 3 micrometers or greater, preferably 5 micrometers or greater, and more preferably 10 micrometers or greater produce larger surface coverage due to cellular growth under both static and flow growth conditions when compared with features having a width of 3 micrometers or less, 2 micrometers or less, and an average feature spacing of 3 micrometers or less, preferably 2 micrometers or less.

The upper spacing limit for individual features for successful growth of cellular matter is 100 micrometers or less, 90 micrometers or less, 80 micrometers or less, 60 micrometers or less, 50 micrometers or less and 30 micrometers or less with individual feature widths of 100 micrometers or less, 90 micrometers or less, 80 micrometers or less, 60 micrometers or less, 50 micrometers or less and 30 micrometers or less. The height of the individual features can be 0.5 to 15 micrometers, preferably 2 to 12 micrometers and preferably 3 to 10 micrometers.

It is to be understood that while the invention has been described in conjunction with the preferred specific embodiments thereof, that the foregoing description as well as the examples, which follow are intended to illustrate and not limit the scope of the invention. Other aspects, advantages and modifications within the scope of the invention will be apparent to those skilled in the art to which the invention pertains.

1. A method of manufacturing a vascular graft comprising:
disposing cells of a first cell type on a core having a textured surface, wherein the textured surface comprises a plurality of spaced features, the spaced features being arranged in a plurality of groupings, and the groupings of the spaced features being arranged with respect to one another so as to define a tortuous path when viewed in a first direction;
growing the cells to form a primary cell-seeded construct, wherein the primary cell-seeded construct has a textured inner surface that is a negative image of the textured surface of the core;
contacting the primary cell-seeded construct with a second cell type to form a secondary cell-seeded construct; and
removing the core to produce the vascular graft.

2. The method of claim 1, wherein the first cell type comprises epithelial cells and the second cell type comprises smooth muscle cells, epithelial cells, macrophages, fibroblasts, or a combination thereof.

3. The method of claim 1, wherein the textured surface comprises a pattern, and wherein a longitudinal axis of the pattern is parallel to a longitudinal axis of the core.

4. The method of claim 1, wherein the core comprises an inflatable bladder or a biodegradable polymer.

5. The method of claim 1, wherein individual spaced features have widths of greater than 3 micrometers to less than 100 micrometers, and wherein spacings between the individual spaced features are greater than 3 micrometers to less than 100 micrometers.

6. The method of claim 1, wherein the core comprises passages that are operative to transmit a temperature controlling fluid that facilitate a contraction of a volume of the core.

7. The method of claim 1, wherein the groupings of spaced features are arranged to define a linear path when viewed in a second direction.

8. The method of claim 7, wherein the plurality of spaced features may be projected outwards from a surface or projected into the surface.

9. The method of claim 1, further comprising decellularizing the vascular graft.

10. The method of claim 1, wherein the primary cell-seeded construct and/or the secondary cell-seeded construct comprises two or more layers.

11. The method of claim 1, wherein the core and the primary cell-seeded construct are biocompatible.

12. The method of claim 1, wherein the primary cell-seeded construct and the secondary cell seeded construct each comprise one or more cell types.

13. The method of claim 1, wherein the primary cell type and the secondary cell type are different from each other and comprise endothelial cells endothelial progenitor cells, smooth muscle cells, macrophages, fibroblasts, or a combination thereof.

14. The method of claim 1, wherein the first cell type and the second cell type are the same.

15. The method of claim 1, where the first cell type and the second cell type are different from each other.

16. A vascular graft comprising:
a primary cell-seeded construct having a texture on its inner surface, wherein the texture comprises a plurality of spaced features, the spaced features being arranged in a plurality of groupings, and the groupings of the spaced features being arranged with respect to one another so as to define a tortuous path when viewed in a first direction; and
a secondary cell-seeded construct disposed on the primary cell-seeded construct.

17. The vascular graft of claim 16, wherein the primary cell-seeded construct comprises two or more layers.

18. The vascular graft of claim 16, further comprising an extracellular matrix.

19. The vascular graft of claim 18, wherein the extracellular matrix comprises a different cell type than a cell type of the primary cell-seeded construct.

20. The vascular graft of claim 18, where the extracellular matrix is an acellular tissue-engineered material.

21. The vascular graft of claim 16, wherein the vascular graft has a tubular cross-sectional area, wherein the textured surface comprises a pattern, and wherein a longitudinal axis of the pattern is parallel to a longitudinal axis of the vascular graft.

22. The vascular graft of claim 16, wherein the vascular graft comprises two or more layers, and wherein an outermost layer comprises a hydrogel, collagen, or a combination thereof.

23. A method of using a vascular graft, the method comprising:
disposing in a body of a living being a vascular graft comprising:
a primary cell-seeded construct having a texture on its inner surface, wherein the texture comprises a plurality of spaced features, the spaced features being arranged in a plurality of groupings, the groupings of spaced features being arranged with respect to one another so as to define a tortuous path when viewed in a first direction; and
a secondary cell-seeded construct disposed on the primary cell-seeded construct, wherein the secondary cell-seeded construct is formed in the body of the living being.
